(19) United States
(12) Patent Application Publication
(10) Pub. No.: US 2009/0299306 A1
(43) Pub. Date: Dec. 3, 2009

(54) Control unit with pump module for a negative pressure wound therapy device

(76) Inventor: John Buan, Maple Grove, MN (US)

Correspondence Address: FACTOR & LAKE, LTD, 1327 W. WASHINGTON BLVD., SUITE 5G/H, CHICAGO, IL, 60607, US

(21) Appl. No.: 12/455,012

(22) Filed: May 27, 2009

Related U.S. Application Data
(60) Provisional application No. 61/128,942, filed on May 27, 2008.

(51) Int. Cl. A61M 1/00

(52) U.S. Cl. 604/319

(57) ABSTRACT

An apparatus including a pump and control unit to be used to provide negative pressure for a wound therapy device. The pump module includes a pump and a pressure sensor, and the control unit module is capable of determining the pump in the pump module. The apparatus has a handheld size.

### RELATED APPLICATIONS

This application claims priority to U.S. Provisional Application 61/128,942 filed on May 27, 2008, the entirety of which is incorporated herein. The invention in the present application relates, generally, in subject matter to the devices disclosed in Applicant's own U.S. Patent Pre-Grant Publication Nos. 2007/0265585 and 2007/0265586.

### TECHNICAL FIELD

The present invention relates, in general, to a device with a pump for providing negative pressure for a wound therapy device that is capable of treating a variety of chronic and acute wound types, including, but not limited to, infection wounds, venous ulcers, arterial ulcers, diabetic ulcers, burn wounds, post amputation wounds, surgical wounds, and the like.

### BACKGROUND

Negative pressure therapy has been one method used for the treatment of a variety of wounds by practitioners in the art. Conventional negative pressure therapy devices are generally large in size and often require the use of complicated equipment such as suction pumps, vacuum pumps and complex electronic controllers.

Some of the currently available negative pressure wound therapy systems do not separate the expensive control and programming portions of the system from the mechanical pumping portion. Thus, in practice, the clinician must send the whole system for sterilization before using the device on a second, or subsequent, patient—as both portions are potentially exposed to bacteria and wound exudates.

Specifically, most commercially available negative pressure wound therapy systems (with the exception of those available from Applicant and described below) are configured to allow exudates and liquid that are removed from the wound to pass outside the dressing to a canister or other structure for retaining the exudates and liquids. If this canister or other structure is incorporated into the apparatus that provides the negative pressure (i.e., the pump), then the apparatus tends to be bulky and rather large and can restrict the movement and mobility of a patient being treated.

In addition, the conventional apparatuses that provide the negative pressure for typical wound therapy devices (e.g., dressings) require a substantial power source such as a constant access to an outlet, or bulky, rechargeable batteries. This is a result of the power needed to run the pump because of the leaking of the dressing that causes dissipation of the negative pressure. Additionally, removal of wound fluid from the dressing requires the pump to be run continuously to maintain negative pressure within the dressing.

Newer wound therapy dressings, such as those described in Applicant's own U.S. Patent Pre-Grant Publication Nos. 2007/0265585 and 2007/0265586, are directed towards retaining the exudates and liquids within the wound therapy dressing itself.

In addition, some of such newer wound therapy dressings are more efficient and less likely to leak or allow the negative pressure to dissipate. For example, in Applicant's own U.S. Provisional Application No. 61/128,957, such a newer dressing is disclosed.

Thus, it would be beneficial to provide an apparatus that provides negative pressure that does not include a canister and does not require a large power source.

In addition, it would be beneficial to provide a device which will allow for a device in which the pump unit may be disposable. Thus, the possibility of cross-contamination between patients is lessened.

### SUMMARY OF THE INVENTION

In accordance with an embodiment of the invention, the invention is a device with a control unit module and a pump module. The control unit module can functionally provide power to the entire system, can allow the user to program the device for therapy and can display relevant operational information including current settings and error messages. The pump module connects at least electrically and mechanically, but not fluidically, with the control unit module and contains the vacuum pump and other support systems. In this way the control unit module is isolated from the functional system plumbing and is unlikely to come in contact with wound exudates and/or bacteria through the pressure hoses that connect to the negative pressure wound dressing. Thus, the control unit module would not need aggressive cleaning when moved to another patient.

In preferred embodiment of the invention, the invention relates to an apparatus to be used to provide negative pressure for a wound therapy dressing. The apparatus may include a pump for creating a negative pressure, a port for providing communication of the negative pressure, and, a power source for the pump, wherein the power source comprises, for example, three AA batteries.

In a further preferred embodiment of the invention, the invention relates to an apparatus to be used to provide negative pressure for a wound therapy dressing. The apparatus may include a pump for creating a negative pressure, a port for providing communication of the negative pressure, a power source for the pump, and a housing containing the pump, the port and the power source, wherein the housing has a handheld size.

In yet another preferred embodiment of the invention, the invention relates to a method for treating a wound with negative pressure. The method generally may include the steps of providing a wound therapy dressing, providing an apparatus including a pump contained in a housing, wherein the housing has a handheld size, and, utilizing the pump to provide a negative pressure, wherein the exudates are retained within the wound therapy dressing.

As will be described in detail below, certain aspects of some of the embodiments of the present invention provide an apparatus including a pump that is handheld. This is believed to be a result of at least two separate advances in the design of negative pressure wound therapy devices.

First, due to an increase in the efficiency of some negative pressure wound therapy dressings, the pumps that provide negative pressure to such dressings do not need such a large power source. It would not have been economically feasible to use commercial disposable (or rechargeable) batteries such as AA, AAA, and the like. The commercial disposable (or rechargeable) batteries would have such a high drain rate, that they would need to be changed constantly. However, because the efficiency of negative pressure wound therapy dressings is increasing, the pump does not require as large of a power source. Accordingly, an apparatus with a pump can be constructed that is handheld, and, that is substantially smaller than conventional pump apparatuses.

Second, due to the types of negative pressure wound therapy dressings wherein exudates are retained within the housing of the wound therapy dressing, the apparatus no longer requires a container or other large structure to collect and retain the exudates and liquid. Thus, the apparatus can be made smaller because it does not require a structure to collect and retain the exudates and liquid.

Thus, based upon the above and other aspects of the invention, the present invention provides one or more embodiments that are handheld. The benefits of having a handheld apparatus include increasing the mobility and movement of a patient being treated with negative pressure wound therapy. A patient could mount such a device to his or her belt and then move around in a relatively normal fashion. In addition, in general, having a smaller apparatus would be beneficial in hospital where storage space may be at a premium.

### BRIEF DESCRIPTION OF THE DRAWINGS

The present invention will become more fully apparent from the following description and appended claims, taken in conjunction with the accompanying drawings. Understanding that the accompanying drawings depict only typical embodiments, and are, therefore, not to be considered to be limiting of the scope of the present disclosure, the embodiments will be described and explained with specificity and detail in reference to the accompanying drawings as provided below.

FIG. 1 is a front view of an apparatus according to the present invention.

FIG. 2 is a back view of an apparatus according to the present invention.

FIG. 3 is a view of a pump module according to the present invention.

FIG. 4 is another view of a pump module according to the present invention.

FIG. 5 is a view of a control unit module according to the present invention.

FIG. 6 is another view of a control unit module according to the present invention.

FIG. 7 is a front perspective view of a belt case to hold an apparatus according to the present invention.

FIG. 8 is a back perspective view of a belt case to hold an apparatus according to the present invention.

### DETAILED DESCRIPTION OF THE PRESENT INVENTION

It will be readily understood that the components of the embodiments as generally described and illustrated in the Figures herein could be arranged and designed in a wide variety of different configurations. Thus, the following more detailed description of various embodiments, as represented in the Figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

In the following description, numerous specific details are provided, such as examples of housings, barriers, chambers etc., to provide a thorough understanding of embodiments of the invention. One skilled in the relevant art will recognize, however, that the invention can be practiced without one or more of the specific details, or with other methods, components, materials, and so forth. In other instances, well-known structures, materials, or operations such as vacuum sources are not shown or described in detail to avoid obscuring aspects of the invention.

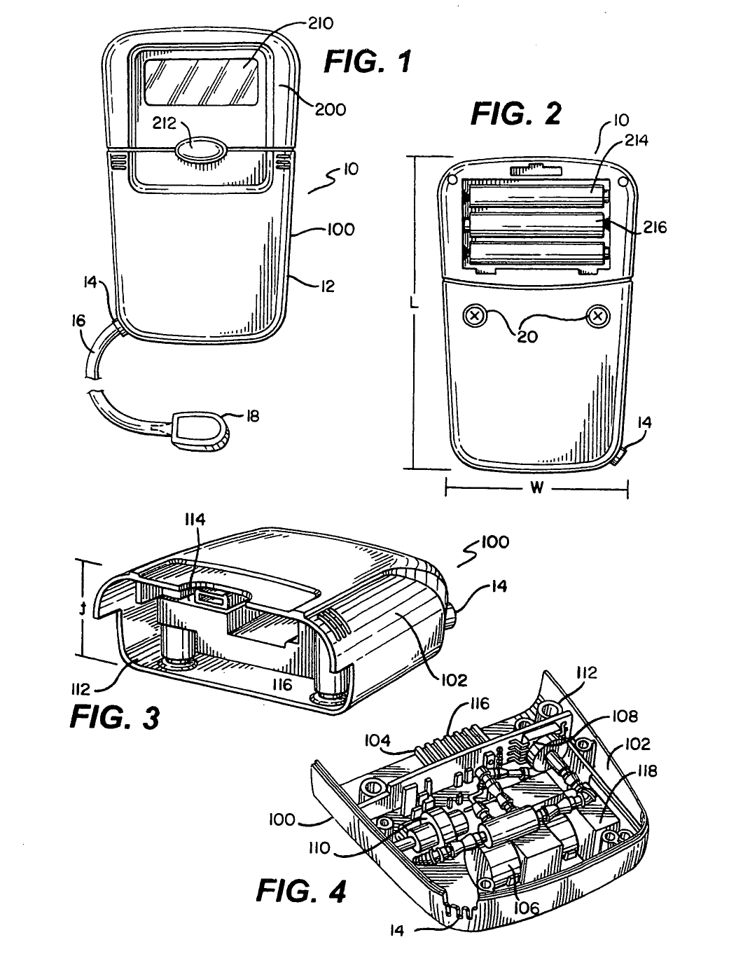

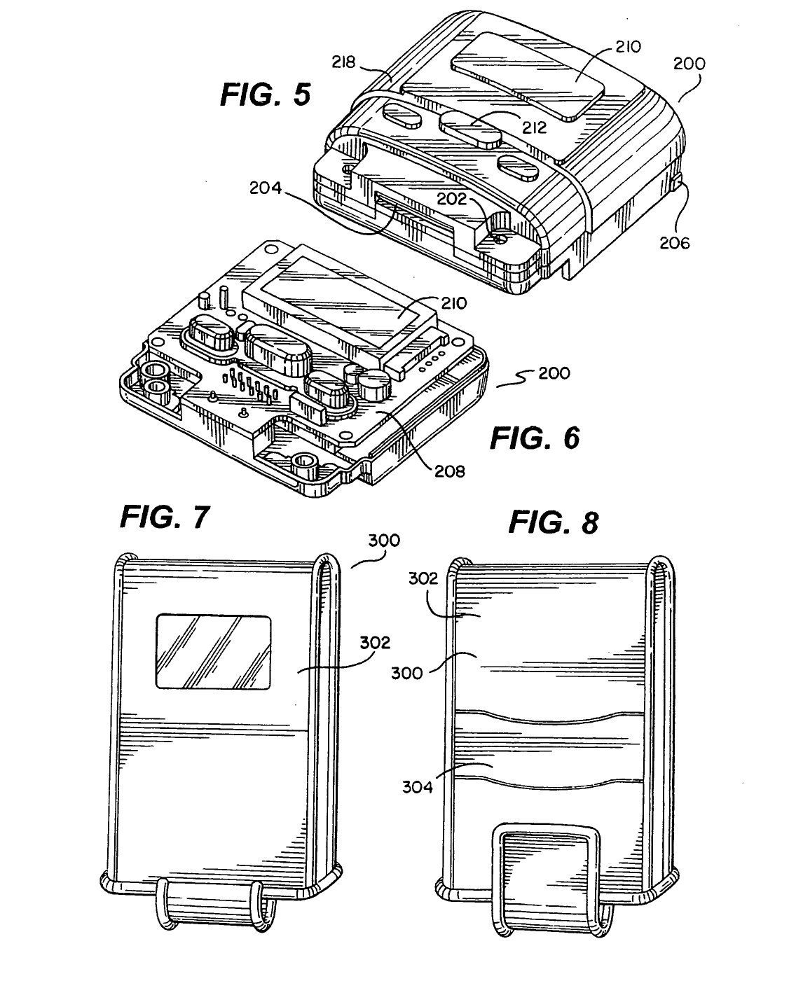

Referring now to FIGS. 1 through 6, an apparatus 10, according to one embodiment of the present invention, generally includes a pump module 100 and a control unit module 200. The apparatus 10 may include a housing 12, or alternatively the pump module 100 may include a housing 102 and the control unit module 200 may include a housing 206. It is preferred that the housing 12 (or alternately the housing 102 and housing 206) is handheld. As used herein, “handheld” is meant to mean that the housing 12 of the apparatus is generally smaller than conventional devices, and, has a length (l) less than approximately nine inches, has a width (w) less than approximately seven inches, and has a thickness (t) less than approximately four inches. In a preferred embodiment, the apparatus has a length (l) of less than or equal to five inches, a width (w) of less than or equal to 3 inches and a thickness (t) of less than or equal to an inch and a half. In addition, a preferred embodiment of the apparatus weighs approximately eight ounces.

The apparatus 10 may also include a port 14. The port 14 may further include tubing 16 and a quick release valve 18. As used herein “quick release valve” is intended to mean a valve or connection point that will allow the apparatus 10 to communicate negative pressure to a negative pressure wound therapy dressing and that includes one or more structures (such as a button) to maintain the connection.

For example, a quick release valve may be a female connector that includes structure to obtain an interference fit with a male connector. The female connector may also include a button that communicates with the structure. When it is desired that the two connectors be disengaged, one need simply depress the button, which would move the structure and disengage the male connector. This description is merely exemplary of a typical quick release valve.

The pump module 100 generally may include a circuit board 104, a pump 106 and a pressure sensor 108. It is also contemplated that the pump module 100 includes a check valve 110 for preventing flow of positive pressure from the pump module 100 to the dressing.

The pump 106 is capable of operating at pressures typical of negative pressure wound therapy, e.g. −25 mmHg to −250 mmHg. Currently, three types of pumps are contemplated to be used with the present invention. A continuous pump is a pump that provides and maintains a desired negative pressure for wound therapy. An intermittent pump is a pump that provides a desired negative pressure for wound therapy and then ceases providing pressure while the negative pressure is released. A third pump, described herein as a “hybrid pump” is a pump capable of operating as either a continuous pump or as an intermittent pump.

In an embodiment with an intermittent pump, it is contemplated that the pump module 100 includes a solenoid valve 118 located between and communicating with the negative wound pressure dressing and the pump. In use, once the intermittent pump has reached the desired pressure, the solenoid valve 118 can open and allow atmospheric air into the negative wound pressure dressing.

In an embodiment with a continuous pump, the pressure sensor 108 can detect a leak in the negative wound pressure dressing and communicate with the pump 106 so that the pump 106 provides the required pressure.

The circuit board 104 can manage and control all electrical aspects of the pump module 100.

The pump module 100 may be removably attached to the control unit module 200. The pump module 100 can be removably attached through any number of mechanical structures, including, but not limited to, tabs, screws, interference members, buttons, clips, snaps, bolts, pins or any other structure that will selectively maintain a connection between the pump module 100 and control unit module 200. In the embodiment depicted screws 20 pass through apertures 112 in the pump module 100 and through a second set of apertures 202 and terminate in screw caps 114 in the pump module 100.

As explained above, the control unit module 200 is isolated, fluidically, from the pump module 100 and specifically from the functional system plumbing and, therefore is unlikely to come in contact with wound exudates and/or bacteria through the pressure hoses that connect to the negative pressure wound dressing. Thus, the control unit module 200 would not need aggressive sterilization when utilized with another patient.

The control unit 200 is also in electronic communication with the pump module 100. In an embodiment shown, the control unit 200 includes a female connection 204 and the pump module 100 includes a male connection 116. When the control unit 200 and pump module 100 are removably attached, the male connection 116 communicates to the female connection 204. Additionally, the electrical connector on the control unit module 200 is preferred to be of the female type to prevent human contact with the power source. Additionally, the electrical connectors, e.g., 204, 116, can also provide the mechanical connection between the pump module 100 and control unit module 200, described above.

The control unit module 200 may also include, generally, a second housing 206, a second circuit board 208, a display 210, a button 212 and a power source 214. The power source 214 can be, but is not limited to, an AA battery 216. Indeed, other conventionally obtained disposable batteries (and smaller rechargeable batteries) are likewise contemplated—including, for example, AAA, A, C, and D sized batteries.

The button 212 can be a power button (i.e., ON/OFF button). Additional buttons can include programming and/or mode selection buttons 218. If the housing 102 of the pump module 100 includes a sliding portion, it is contemplated that the sliding portion, in a closed configuration, cover one of more of the programming and/or mode selection buttons 218. The user can manipulate the sliding portion to an open position to access the buttons 218.

The display 210 can be an LCD display or an LED display, or any other display.

The second circuit board 208 is capable of detecting the type of pump 106 that is located in the pump module 100. One method that can be used for detecting the type of pump involves the use of resistors. For example, the second circuit board 208 can be pre-programmed with a specific resistance value for each of the three types of pumps described above (continuous, intermittent, or a pump capable of both). When the pump module 100 is connected to the control unit module 200 (since they are in electrical communication), the second circuit board 208 can detect the resistance of the pump. The second circuit board 208 can then communicate with the pump 106 allowing the pump to function appropriately. Additionally if the pump is one that is capable of functioning as either a continuous pump or an intermittent pump, when the second circuit board 208 detects the pump, the control unit can give the user the option of selecting the type of pump therapy to be used. For example, the display may display a message, and the user can depress a button to select the type of pump therapy to use.

The above described embodiments relate to the present invention as an apparatus. As previously mentioned an embodiment of the present invention of is directed to a method for treating a wound with negative pressure.

Generally, this method includes the steps of providing a wound therapy dressing, providing an apparatus including a pump contained in a housing, wherein the housing has a handheld size, and, utilizing the pump to provide a negative pressure, wherein the exudates are retained within the wound therapy dressing. For this embodiment of the invention, it is preferred that the apparatus be the one described herein. Moreover, it is preferred, but not required, that the wound therapy dressing be one described in Applicant's own U.S. Patent Pre-Grant Publication Nos. 2007/0265585 and 2007/0265586. However, nothing herein should be construed as limited this embodiment, or any other embodiment, to these specific types of wound therapy dressings.

This embodiment may also include the step of selecting the mode of operation of the pump. This step is intended, but not required, to be utilized when the pump is a hybrid pump.

Furthermore, this embodiment may include the step of mounting the apparatus to a belt. FIGS. 7 and 8 depict a means for mounting the apparatus to a belt 300. The means for mounting 300 can be, for example, a pouch 302 with a belt loop 304. Other means for mounting may include, loops, clips, snaps, cases, clasp, hooks, molded cases, hook and loop fasteners, button, zipper, magnet, and other similar structures.

Without further elaboration, it is believed that one skilled in the art can use the preceding description to utilize the present disclosure to its fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles of the disclosure provided herein. In other words, various modifications and improvements of the embodiments specifically disclosed in the description above are within the scope of the appended claims. Note that elements recited in means-plus-function format are intended to be construed in accordance with 35 U.S.C. § 112 ¶6. The scope of the invention is therefore defined by the following claims.

1. An apparatus to be used to provide negative pressure for a wound therapy device, the apparatus comprising:
a pump module comprising:
a pump wherein the pump is selected from the group consisting of: a continuous pump, an intermittent pump, and a hybrid pump; and,
a pressure sensor; and,
a control unit module in electrical communication with the pump module and capable of determining the pump in the pump module.

2. The apparatus of claim 1 further comprising a port for communicating a negative pressure.

3. The apparatus of claim 2 wherein the port further comprises a tubing and a quick release valve.

4. The apparatus of claim 1 further comprising a power source.

5. The apparatus of claim 4 wherein the power source comprises at least two AA batteries.

6. The apparatus of claim 1 further comprising a housing wherein the housing has a handheld size.

7. An apparatus to be used to provide negative pressure for a wound therapy device, the apparatus comprising:
a pump for creating a negative pressure;
a port for providing communication of the negative pressure; and,
a power source for the pump, wherein the power source comprises three AA batteries.

8. The apparatus of claim 7, wherein the pump is selected from the group consisting of: a continuous pump, an intermittent pump, and a hybrid pump.

9. The apparatus of claim 8 further comprising a housing wherein the housing has a handheld size.

10. The apparatus of claim 7 further comprising a tubing communicating with the port and a quick release valve disposed at an end of the tubing.

11. An apparatus to be used to provide negative pressure for a wound therapy device, the apparatus comprising:
a pump for creating a negative pressure;
a port for providing communication of the negative pressure;
a power source for the pump; and,
a housing containing the pump, the port and the power source, wherein the housing has a handheld size.

12. The apparatus of claim 11 further comprising the pump being selected from the group consisting of: a continuous pump, an intermittent pump, and a hybrid pump.

13. The apparatus of claim 12 further comprising a tubing communicating with the port and a quick release valve disposed at an end of the tubing.

14. The apparatus of claim 12 further comprising a control unit module in electrical communication with the pump and capable of determining the pump.

15. The apparatus of claim 11 wherein the housing has a length approximately five inches, a width approximately three inches and a thickness approximately an inch and a half.

16. The apparatus of claim 15 wherein the apparatus has an overall weight of eight ounces.

17. The apparatus of claim 11 wherein the housing has a length less than approximately nine inches, a width less than approximately seven inches and a thickness less than approximately four inches.

18. The apparatus of claim 11 further comprising an LCD display.

19. The apparatus of claim 11 further comprising a means for mounting the apparatus to a belt.

20. A method for treating a wound with negative pressure, the method comprising the steps of:
providing a wound therapy dressing;
providing an apparatus including a pump contained in a housing, wherein the housing has a handheld size; and,
utilizing the pump to provide a negative pressure, wherein the exudates are retained within the wound therapy dressing.

21. The method of claim 20 wherein the pump is a hybrid pump and further comprising the step of:
selecting the mode of operation of the pump.

22. The method of claim 20 further comprising the step of:
mounting the apparatus to a belt.
